(19) United States
(12) Patent Application Publication
(10) Pub. No.: US 2025/0041552 A1
(43) Pub. Date: Feb. 6, 2025

(54) Injectable Medical Formulation for Spinal Anesthesia, Process of Preparing, and Process of Administering

(71) Applicant: Kimberly Babiash, Wichita, KS (US)

(72) Inventor: Kimberly Babiash, Wichita, KS (US)

(21) Appl. No.: 18/792,963

(22) Filed: Aug. 2, 2024

Related U.S. Application Data
(60) Provisional application No. 63/517,288, filed on Aug. 2, 2023.
(60) Provisional application No. 63/587,247, filed on Oct. 2, 2023.
(60) Provisional application No. 63/625,669, filed on Jan. 26, 2024.

(51) Int. Cl. A61M 19/00; A61J 1/20; A61K 9/00; A61K 31/137; A61K 31/4164; A61K 31/4168; A61K 31/445; A61K 31/4468; A61K 31/4535; A61K 31/485

(52) U.S. Cl. CPC A61M 19/00; A61J 1/2093; A61J 1/2096; A61K 9/0019; A61K 31/137; A61K 31/4164; A61K 31/4168; A61K 31/445; A61K 31/4468; A61K 31/4535; A61K 31/485; A61M 2202/048; A61M 2210/1003

(57) ABSTRACT

A combination of a container and an injectable medical formulation for use in spinal anesthesia as part of a medical procedure includes the injectable medical formulation including a pre-determined amount of a local anesthetic; a pre-determined amount of a short-acting opioid, the short-acting opioid being one of fentanyl or sufentanil; and a pre-determined amount of a long-acting opioid, the long-acting opioid being one of morphine and hydromorphone. The container is a single apparatus in which the injectable medical formulation is packaged and stored as a single dose prior to the medical procedure. The injectable medical formulation is packaged and stored within the container prior to a window of imminency of the medical procedure.

### CROSS-REFERENCT TO RELATED APPLICATIONS

This application claims priority to U.S. Provisional Application No. 63/517,288, filed Aug. 2, 2023; U.S. Provisional Application No. 63/587,247, filed Oct. 2, 2023; and U.S. Provisional Application No. 63/625,669, filed Jan. 26, 2024, all of which are incorporated by reference in their entirety herein.

### BACKGROUND

Embodiments of the disclosure relate to anesthetic preparations, and in particular to an injectable medical formulation for spinal anesthesia prepared and processed in advance of a medical procedure, a process of preparing the injectable medical formulation, and a process of administering the injectable medical formulation.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

In some aspects, the techniques described herein relate to a combination of a container and an injectable medical formulation for use in spinal anesthesia as part of an obstetric procedure, the combination including: the injectable medical formulation including: a pre-determined amount of a local anesthetic, the local anesthetic being a hyperbaric bupivacaine; a pre-determined amount of a short-acting opioid, the short-acting opioid being one of fentanyl or sufentanil; and a pre-determined amount of a long-acting opioid, the long-acting opioid being one of morphine or hydromorphone; the container being a single apparatus in which the injectable medical formulation is packaged and stored as a single dose prior to the obstetric procedure; wherein the injectable medical formulation is packaged and stored within the container prior to a window of imminency of the obstetric procedure.

In some aspects, the techniques described herein relate to a combination, wherein the injectable medical formulation is packaged and stored within the container to create the single dose prior to knowledge of a specific obstetric procedure for which the single dose will be used.

In some aspects, the techniques described herein relate to a combination, wherein the container consists of a vial in which the single dose is stored prior to the obstetric procedure, the single dose stored in such a way that requires one withdrawal from the vial for administration to a patient.

In some aspects, the techniques described herein relate to a combination, wherein the container consists of a syringe in which the single dose is stored prior to the obstetric procedure, the single dose stored in such a way that requires one injection into a patient for administration of the single dose.

In some aspects, the techniques described herein relate to a combination, wherein the container includes a plurality of chambers, each chamber holding one or more parts of the single dose, the container configured to facilitate mixing of the one or more parts to create the single dose prior to administration to a patient but without any measuring of the one or more parts.

In some aspects, the techniques described herein relate to a combination, wherein the injectable medical formulation further includes: the pre-determined amount of the local anesthetic and the local anesthetic are selected from a group consisting of: the local anesthetic being 0.75% hyperbaric bupivacaine and the predetermined amount of the local anesthetic being an amount between approximately 10.5 mg and 12.0 mg; and the local anesthetic being 0.5% hyperbaric bupivacaine and the pre-determined amount of the local anesthetic being an amount between approximately 10.5 mg and 12.5 mg; the pre-determined amount of the short-acting opioid and the short-acting opioid are selected from a group consisting of: the short-acting opioid being fentanyl at a concentration of 50 mcg/mL and the pre-determined amount of the short-acting opioid being an amount between approximately 5 mcg and 50 mcg; and the short-acting opioid being sufentanil and the pre-determined amount of the short-acting opioid being an amount between approximately 5 mcg and 20 mcg; the pre-determined amount of the long-acting opioid and the long-acting opioid are selected from a group consisting of: the long-acting opioid being morphine at a concentration of 0.5 mg/mL and the pre-determined amount of the long-acting opioid being an amount between approximately 50 mcg and 300 mcg; and the long-acting opioid being hydromorphone and the pre-determined amount of the long-acting opioid being an amount between approximately 25 mcg and 100 mcg; wherein the obstetric procedure is a cesarian section.

In some aspects, the techniques described herein relate to a combination, wherein the injectable medical formulation further includes one or more adjuvant medications, the one or more adjuvant medications being added to the injectable medical formulation at pre-determined amounts, the one or more adjuvant medications in pre-determined amounts being selected from a group consisting of: an amount between 5 mcg and 25 mcg of dexmedetomidine; an amount between 5 mcg and 25 mcg of clonidine; and an amount between 5 mcg and 250 mcg of epinephrine.

In some aspects, the techniques described herein relate to a combination, wherein the injectable medical formulation further includes: the pre-determined amount of the local anesthetic and the local anesthetic are selected from a group consisting of: the local anesthetic being 0.75% hyperbaric bupivacaine and the predetermined amount of the local anesthetic being an amount between approximately 11.25 mg and 12 mg; and the local anesthetic being 0.5% hyperbaric bupivacaine and the pre-determined amount of the local anesthetic being an amount between approximately 11.25 mg and 12.5 mg; the short-acting opioid is fentanyl at a concentration of 50 mcg/mL and the pre-determined amount of the short-acting opioid is an amount between approximately 10 mcg and 20 mcg; and the long-acting opioid is morphine at a concentration of 0.5 mg/mL and the predetermined amount of the long-acting opioid is an amount between approximately 50 mcg and 150 mcg; and wherein the obstetric procedure is a cesarian section.

In some aspects, the techniques described herein relate to a combination, wherein the injectable medical formulation further includes: the pre-determined amount of the local anesthetic and the local anesthetic are selected from a group consisting of: the local anesthetic being 0.75% hyperbaric bupivacaine and the predetermined amount of the local anesthetic being approximately 7.5 mg; and the local anesthetic being 0.5% hyperbaric bupivacaine and the pre-determined amount of the local anesthetic being an approximately 7.5 mg; the pre-determined amount of the short-acting opioid and the short-acting opioid are selected from a group consisting of: the short-acting opioid being fentanyl at a concentration of 50 mcg/mL and the pre-determined amount of the short-acting opioid being an amount between approximately 10 mcg and 20 mcg; and the short-acting opioid being sufentanil and the pre-determined amount of the short-acting opioid being an amount between approximately 5 mcg-20 mcg; the pre-determined amount of the long-acting opioid and the long-acting opioid are selected from a group consisting of: the long-acting opioid being morphine at a concentration of 0.5 mg/mL and the pre-determined amount of the long-acting opioid being an amount between approximately 50 mcg and 150 mcg; and the long-acting opioid being hydromorphone and the pre-determined amount of the long-acting opioid being an amount between approximately 25 mcg and 100 mcg; wherein the obstetric procedure is a procedure after a failed labor epidural.

In some aspects, the techniques described herein relate to a combination, wherein the injectable medical formulation further includes one or more adjuvant medications, the one or more adjuvant medications being added to the injectable medical formulation at pre-determined amounts, the one or more adjuvant medications in pre-determined amounts being selected from a group consisting of: an amount between 5 mcg and 25 mcg of dexmedetomidine; an amount between 5 mcg and 25 mcg of clonidine; and an amount between 5 mcg and 250 mcg of epinephrine.

In some aspects, the techniques described herein relate to a combination, wherein the injectable medical formulation further includes: the pre-determined amount of the local anesthetic and the local anesthetic are selected from a group consisting of: the local anesthetic being 0.75% hyperbaric bupivacaine and the predetermined amount of the local anesthetic being approximately 7.5 mg; and the local anesthetic being 0.5% hyperbaric bupivacaine and the pre-determined amount of the local anesthetic being approximately 7.5 mg; the short-acting opioid is fentanyl at a concentration of 50 mcg/mL and the pre-determined amount of the short acting opioid is an amount between approximately 10 mcg and 20 mcg; and the long-acting opioid is morphine at a concentration of 0.5 mg/mL and the predetermined amount of the long-acting opioid is an amount between approximately 50 mcg and 150 mcg; wherein the obstetric procedure is a procedure after a failed labor epidural.

In some aspects, the techniques described herein relate to a combination of a container and an injectable medical formulation for use in spinal anesthesia as part of an orthopedic procedure, the combination including: the injectable medical formulation including: a pre-determined amount of a local anesthetic; a pre-determined amount of a short-acting opioid, the short-acting opioid being one of fentanyl or sufentanil; and a pre-determined amount of a long-acting opioid, the long-acting opioid being one of morphine or hydromorphone; the container being a single apparatus in which the injectable medical formulation is packaged and stored as a single dose prior to the orthopedic procedure; wherein the injectable medical formulation is packaged and stored within the container prior to a window of imminency of the orthopedic procedure.

In some aspects, the techniques described herein relate to a combination, wherein the injectable medical formulation is packaged and stored within the container to create the single dose prior to knowledge of a specific orthopedic procedure for which the single dose will be used.

In some aspects, the techniques described herein relate to a combination, wherein the container consists of a vial in which the single dose is stored prior to the orthopedic procedure, the single dose stored in such a way that requires one withdrawal from the vial for administration to a patient.

In some aspects, the techniques described herein relate to a combination, wherein the container consists of a syringe in which the single dose is stored prior to the orthopedic procedure, the single dose stored in such a way that requires one injection into a patient for administration of the single dose.

In some aspects, the techniques described herein relate to a combination, wherein the container includes a plurality of chambers, each chamber holding one or more parts of the single dose, the container configured to facilitate mixing of the one or more parts to create the single dose prior to administration to a patient but without any measuring of the one or more parts.

In some aspects, the techniques described herein relate to a combination, wherein the injectable medical formulation further includes: the pre-determined amount of the local anesthetic and the local anesthetic being selected from a group consisting of: the local anesthetic being 0.75% hyperbaric bupivacaine and the pre-determined amount of the local anesthetic being approximately 2 mL; the local anesthetic being 0.5% hyperbaric bupivacaine and the pre-determined amount of the local anesthetic being approximately 3 mL; the local anesthetic being 1.5% mepivacaine and the pre-determined amount of the local anesthetic being an amount between approximately 3 mL and 4 mL; the local anesthetic being 0.5% ropivacaine and the pre-determined amount of the local anesthetic being an amount between approximately 2.5 mL and 4 mL; the local anesthetic being 0.5% bupivacaine isobaric and the pre-determined amount of the local anesthetic being an amount between approximately 2 mL and 3 mL; the local anesthetic being 2% mepivacaine and the pre-determined amount of the local anesthetic being an amount between approximately 2 mL and 3 mL; and the local anesthetic being 0.5% hyperbaric ropivacaine and the pre-determined amount of the local anesthetic being an amount between approximately 2.5 mL and 4 mL; the pre-determined amount of the short-acting opioid and the short-acting opioid being selected from a group consisting of: the short-acting opioid being fentanyl at a concentration of 50 mcg/mL and the pre-determined amount of the short-acting opioid being an amount between approximately 5 mcg and 50 mcg; and the short-acting opioid being sufentanil and the pre-determined amount of the short-acting opioid being an amount between approximately 5 mcg and 20 mcg; the pre-determined amount of the long-acting opioid and the long-acting opioid being selected from a group consisting of: the long-acting opioid being morphine at a concentration of 0.5 mg/mL and the pre-determined amount of the long-acting opioid being an amount between approximately 50 mcg and 300 mcg; and the long-acting opioid being hydromorphone and the pre-determined amount of the long-acting opioid being an amount between approximately 25 mcg and 100 mcg.

In some aspects, the techniques described herein relate to a combination, wherein the injectable medical formulation further includes one or more adjuvant medications, the one or more adjuvant medications being added to the injectable medical formulation at pre-determined amounts, the one or more adjuvant medications in pre-determined amounts being selected from a group consisting of: an amount between 5 mcg and 25 mcg of dexmedetomidine; an amount between 5 mcg and 25 mcg of clonidine; and an amount between 5 mcg and 250 mcg of epinephrine.

In some aspects, the techniques described herein relate to a combination, wherein the injectable medical formulation further includes: the pre-determined amount of the local anesthetic and the local anesthetic are selected from a group consisting of: the local anesthetic being 0.75% hyperbaric bupivacaine and the predetermined amount of the local anesthetic being approximately 15 mg; the local anesthetic being 0.5% hyperbaric bupivacaine and the pre-determined amount of the local anesthetic being approximately 15 mg; the local anesthetic being 1.5% mepivacaine and the pre-determined amount of the local anesthetic being an amount between approximately 45 mg and 60 mg; the local anesthetic being 0.5% ropivacaine and the pre-determined amount of the local anesthetic being an amount between approximately 12.5 mg and 20 mg; the local anesthetic being 0.5% bupivacaine isobaric and the pre-determined amount of the local anesthetic being an amount between approximately 10 mg and 15 mg; the local anesthetic being 2% mepivacaine and the pre-determined amount of the local anesthetic being an amount between approximately 40 mg and 60 mg; and the local anesthetic being 0.5% hyperbaric ropivacaine and the pre-determined amount of the local anesthetic being an amount between 12.5 mg and 20 mg; the short-acting opioid is fentanyl at a concentration of 50 mcg/mL and the pre-determined amount of the short-acting opioid is an amount between approximately 10 mcg and 20 mcg; and the long-acting opioid is morphine at a concentration of 0.5 mg/mL and the predetermined amount of the long-acting opioid is an amount between approximately 50 mcg and 150.

In some aspects, the techniques described herein relate to a process of packaging an injectable medical formulation for use in spinal anesthesia, the process including: providing: (i) a pre-determined amount of a local anesthetic; (ii) a pre-determined amount of a short-acting opioid, the short-acting opioid being one of fentanyl or sufentanil; and (ii) a pre-determined amount of a long-acting opioid, the long-acting opioid being one of morphine and hydromorphone; packaging the predetermined amount of the local anesthetic, the pre-determined amount of the short-acting opioid, and the pre-determined amount of the long-acting opioid into a container as a single dose; sealing the pre-determined amount of the local anesthetic, the pre-determined amount of the short-acting opioid, and the pre-determined amount of the long-acting opioid into the container; and configuring the container for transport to a medical treatment facility for a later administration of the single dose as part of a medical procedure, the medical procedure being one of: (i) an obstetric procedure; or (ii) an orthopedic procedure.

In some aspects, the techniques described herein relate to a process including: executing the packaging and sealing of the pre-determined amount of the local anesthetic, the pre-determined amount of the short-acting opioid, and the pre-determined amount of the long-acting opioid into the container, and the configuring of the container for transport at a location separate from the medical facility and prior to knowledge of the medical procedure.

In some aspects, the techniques described herein relate to a process including: selecting a vial to serve as the container; configuring the vial to require withdrawal from the vial for administration to a patient.

In some aspects, the techniques described herein relate to a process including: providing a syringe to serve as the container.

In some aspects, the techniques described herein relate to a process including: including a plurality of chambers in the container; holding one or more parts of the single dose in the each of the chambers such that an actuation of a syringe results in a mixing of the one or more parts to create the single dose prior to or during the administration to a patient without any measuring of the one or more parts.

In some aspects, the techniques described herein relate to a process including: selecting hyperbaric bupivacaine to serve as the local anesthetic.

### BRIEF DESCRIPTION OF THE DRAWING FIGURES

Embodiments of the invention are described in detail below with reference to the attached drawing figures, wherein:

FIG. 1 is a block diagram of a process of preparing an injectable medical formulation in accordance with the present invention.

FIG. 2 is a flowchart of the process of preparing the injectable medical formulation of FIG. 1; and

FIG. 3 is a flowchart of a process of administering the injectable medical formulation of FIG. 1.

The drawing figures do not limit the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the invention is defined only by the appended claims, along with the full scope of the equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the technology can include a variety of combinations and/or integrations of the embodiments described herein.

The science of practicing anesthesiology involves utilizing medications to alter one's state of consciousness while modulating pain perception. Neuraxial anesthesia comprises a specialized anesthetic where medications are administered via either the epidural route or the intrathecal route to block neural transmission of pain. Spinal anesthesia is commonly used for a variety of lower body procedures, such as cesarean sections, birth after a failed labor epidural in pregnant women, and orthopedic surgery, such as joint replacements. Spinal anesthesia involves the deposition of a medication specially formulated for intrathecal administration. Intrathecal refers to the space inside the spine where the spinal fluid bathes the spinal cord. Placing medication directly into the spinal fluid allows for a quick onset of surgical anesthesia. This process is considered the industry standard for cesarean sections and is increasingly becoming the industry standard for joint replacements and other procedures. Medications that are intended for the central nervous system, specifically epidurals and spinals, must be specifically formulated for this space, and commonly said medications are preservative free.

The need for administering a spinal anesthetic can arise quickly in an urgent or routine procedure. For example, a complication during labor can require an immediate cesarean section to protect the mother and child. Accordingly, time can be of the essence to prepare the patient for the procedure.

One conventional process of preparing and administering a spinal anesthetic involves separately drawing different formulation components from separate vials in preparation for making an injection into a patient at the time of the medical procedure. These separately drawn components form an aggregation which is administered as the spinal anesthetic. The separate medications are typically separately drawn at the time of surgery or procedure at a patient location, e.g., a treating facility. One multi-step process for a scheduled cesarean section includes: (1) withdrawing 1.6 mL of 0.75% hyperbaric bupivacaine into a 3 mL syringe; (2) withdrawing 0.2 mL of fentanyl from a fentanyl vial (containing 2.0 mL of fentanyl 50 mcg/mL) with a tuberculin syringe, thus wasting the remaining 1.8 mL; (3) withdrawing 0.2 mL of preservative-free morphine from a the preservative-free morphine vial (containing 10 mL morphine 0.5 mg/mL) with a second tuberculin syringe, thus wasting the remaining 9.8 mL; (4) adding the 0.2 mL of fentanyl to the 3 mL syringe containing the hyperbaric bupivacaine; and (5) adding the 0.2 mL of preservative-free morphine to the 3 mL syringe containing the fentanyl and hyperbaric bupivacaine to complete the final formulation for injection (in this case 12 mg hyperbaric bupivacaine, 10 mcg fentanyl, and 100 mcg preservative free morphine). In some conventional processes, additional medications, referred to as adjuvant medications, are also added, requiring another mixing step prior to administering the anesthetic to the patient. This multi-step process requires time and precision and can easily result in human error due to the numerous steps, which in many scenarios take place under the stress of an urgent procedure, such as a cesarean section, or a busy ambulatory surgery center/joint hospital. Human error in this specialized practice can result in infection and or misdoing, leading to severe morbidity and even mortality. In addition, these conventional processes result in wasted medications, specifically wasted narcotics which are highly regulated due to the risk of diversion and misuse. Thus, the conventional spinal anesthetic processes are wasteful, require specialization in dosing, are time-consuming, are fraught with error and provide opportunity for diversion.

Accordingly, the disclosed process embodiments provide an injectable medical formulation for spinal anesthesia prepared and processed in advance of a medical procedure, a process of preparing the injectable medical formulation, and a process of administering the injectable medical formulation. In more specific embodiments, the injectable medical formulation is preliminarily prepared at a compounding or other facility located remotely relative to the patient and/or treatment facility. This ensures that the anesthesiologist administering the formulation is not required to measure and mix multiple medications on-site at the time of the medical procedure. The process of preparing the injectable medical formulation includes embodiments wherein an appropriate combination of medications is mixed and then held in a single container at a first location, such as in a pharmaceutical lab, before being provided to a medical facility. In addition, embodiments of the process of administering the injectable medical formulation involve little to no mixing and measuring steps at the treatment facility, such that the potential for human error is reduced, efficiency is increased, risks of contamination are reduced, and potential abuse of controlled substances in this setting is eliminated.

Those skilled in the art will appreciate that the injectable medical formulations disclosed herein, the process of preliminary remote mixing and containment, and the process of administering, may be applied to a plurality of medical procedures including conventional, routine, and critical scenarios. Examples discussed herein include spinal anesthesia as part of obstetric procedures, such as cesarian sections and procedures after a failed epidural, and orthopedic procedures. However, the methodologies and formulations can be applied to any gynecological procedure, lower extremity procedure, and any procedure considered underneath the umbilicus. Orthopedic procedures include any procedure that conventionally utilizes spinal anesthesia as part of an orthopedic surgery in adults.

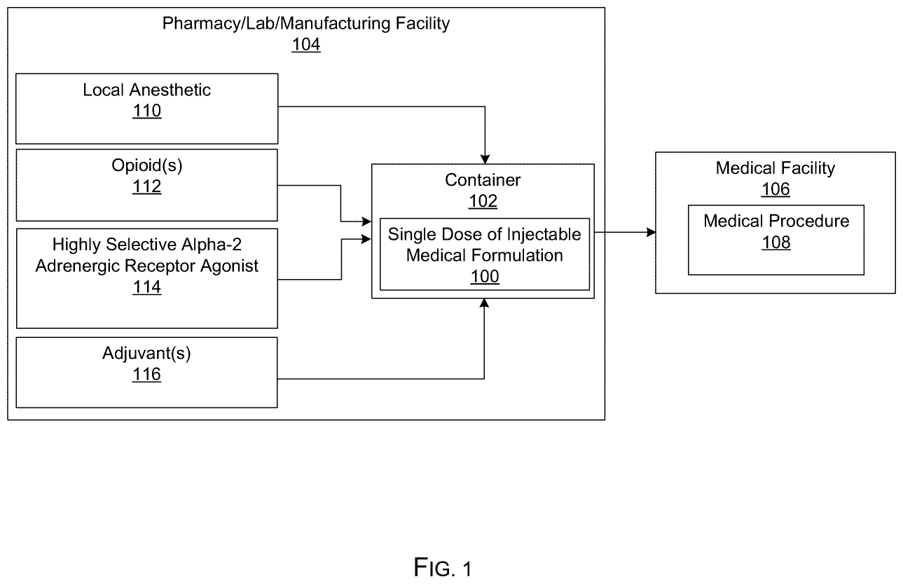

In FIG. 1 a simplified block diagram depicts a method of preparing a single dose of an injectable medical formulation 100 within a single container 102. As shown, the formulation 100 is prepared, packaged, contained, sealed, and stored within the single container 102 at a time and place separate from where a medical procedure 108 is taking place. For example, the formulation 100 may be prepared in a manufacturing facility 104 suitable for medical preparations. For example, the manufacturing facility 104 may be a lab or pharmacy, independent or adjoined with a medical facility 106 where the medical procedure 108 is performed. In accordance with the invention, it is important that the single dose of the injectable medical formulation 100 is prepared and packaged at a separate time from the medical procedure 108. For example, a plurality of single containers 102, each having a single dose of the formulation 100, may be prepared, contained, packaged, and sealed within a container in the facility 104. Here, packaging, containing, and sealing means that the single dose is packaged and sealed within a container such that the single dose remains within the container as prepared until needed for a medical procedure. Once contained at the facility 104, the container (optionally along with other like containers) is then shipped or transported to the medical facility 106 for storage until needed, or immediate use in alternative embodiments. Appropriate storage times may vary based on the specific formulation and technology requirements. For example, the formulation may be developed such that long term storage (days/weeks/months) is appropriate, or short-term storage (hours) is appropriate.

The preparation of the medical formulation 100 takes place in a time period outside of a window of imminency of the medical procedure 108. The window of imminency is a time period occurring immediately prior to the medical procedure 108 in which simultaneous medical preparations for the medical procedure 108 are occurring. In some cases, the time period happens in a state of urgency. In other embodiments, the preparation of the medical formulation 100 takes place in a time period before knowledge of a specific medical procedure 108. In other words, the preparation of the formulation 100 may occur without the single container 102 being specifically intended for a specific medical procedure. In each embodiment, the medical formulation 100 is prepared in advance, such that at the time of the medical procedure 108, the formulation 100 is already prepared and ready for administration. Advanced preparation reduces the risks, discussed above, that are associated with preparing the formulation at the time of the procedure.

As further shown in FIG. 1, the container 102 will receive medications appropriate to create the medical formulation 100. In embodiments, these medications include a local anesthetic 110, one or more opioids 112, an optional highly selective alpha-2 adrenergic receptor agonist 114, and one or more additional optional adjuvant medications 116. Pre-determined and pre-measured amounts of each selected medication are added to the single container 102, resulting in the single dose of the injectable medical formulation 100. Accordingly, at the time of use, the formulation 100 requires little to no measuring or mixing by the medical personnel, reducing the risk for human error.

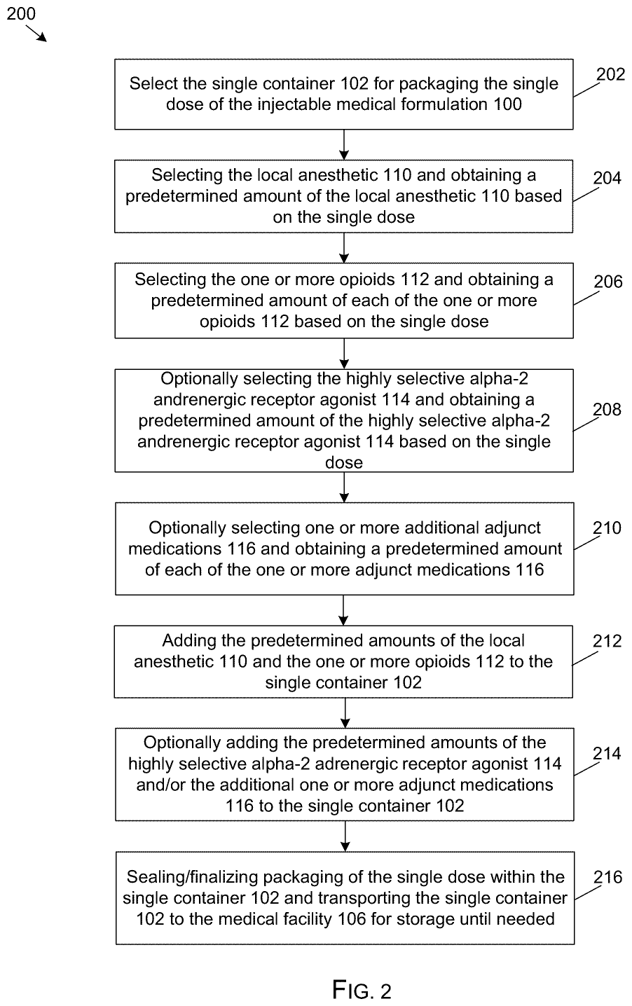

In FIG. 2, a flowchart 200 further depicts the process of preparing the injectable medical formulation 100. At step 202, the single container 102 is selected. The single container 102 may be selected as would be understood by those skilled in the art, such as a single dose glass ampule, a single dose vial, or a single dose glass or synthetic syringe. In some embodiments, the single container 102 holds multiple chambers, such that each medication is pre-measured and held within a respective individual chamber until time of withdraw or injection.

At step 204, the local anesthetic 110 is selected and a predetermined amount is obtained based on the single dose. In other words, an artisan will select and obtain a predetermined amount based on calculations necessary to create the single dose, the calculations varying based on the surgical procedure for which the formulation is intended.

At step 206, the one or more opioids 112 are selected and a predetermined amount of each is obtained, again the amounts being selected based on calculations for the single dose indicated for the procedure.

At step 208, in some embodiments, the highly selective apha-2 adrenergic receptor agonist 114 is selected and a predetermined amount obtained based on calculations for the single dose. This step is optional as not all formulations will include the highly selective apha-2 adrenergic receptor agonist 114.

At step 210, in some embodiments, one or more additional adjuvant medications 116 are selected and predetermined amounts obtained based on calculations for the single dose.

At step 212, the predetermined amounts of the local anesthetic 110 and the one or more opioids 112 are added to the single container 102. In this step, they are either mixed or added to separate chambers within the single container 102. At step 214, the optional medications, namely the highly selective alpha-2 adrenergic receptor agonist 114 and the one or more additional adjuvant medications 116, are added to the single container 102, again either mixed into the full formulation, or added into individual chambers within the container 102, which can be mixed at time of injection or withdraw.

At step 216, once all selected medications are added to the single container 102, the container 102 is packaged and/or sealed and/or finalized for storage and/or transportation. In other words, the single container 102 with the single dose of the injectable medical formulation 100 is considered completed and will remain stored until needed for a medical procedure.

Subsequent to the FIG. 2 processes, a transportation step occurs wherein one or more sealed and packaged containers are moved from the manufacturing facility 104 via some form of transportation (e.g., mail, courier) to the medical facility 106.

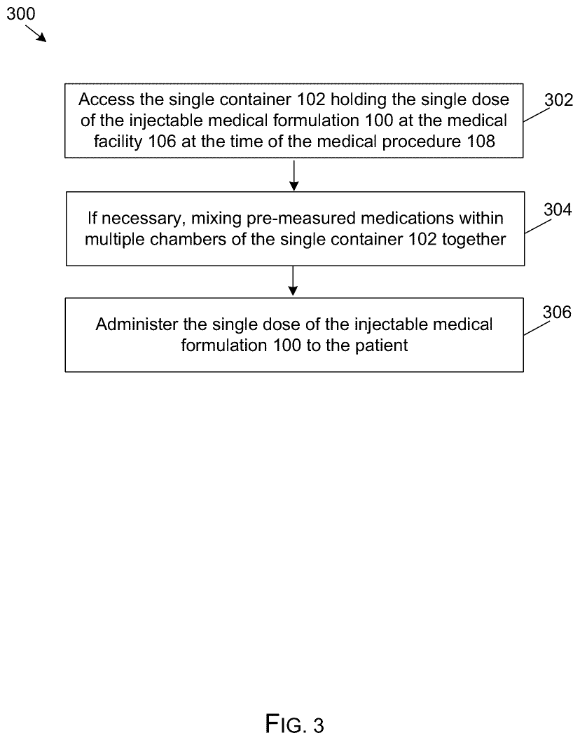

Now turning to FIG. 3, another flowchart depicts the process 300 of administering the injectable medical formulation 100. At step 302, since the single container 102 is previously prepared, the medical personnel will access the single container 102 holding the single dose of the injectable medical formulation 100 at the medical facility 106 and at the time of the medical procedure 108.

At step 304, in embodiments wherein the single container 102 is multi-chambered, the medical personnel will mix the medications in each chamber together to create a final product, such as by puncturing seals. In such embodiments, the medications in each chamber are pre-measured, and accordingly, the medical professional is merely mixing, without necessary measuring steps. The chambers may be sealed using any material that is sufficient to seal the chamber but that can be punctured or manually removed to facilitate mixing of the medications. In some embodiments, the multi-chambered single container 102 will be configured such that merely injecting/administering the formulation 100 will cause the medications to mix such that no specific mixing steps are required. This might be done by the establishment of rupturable barriers being established which may be disrupted by the person administering the dose into a patient to create a local mixing of the components. This may be, in embodiments, accomplished in a multicompartment syringe, vial, or some other sort of container enabling on the spot mixing of the components.

At step 306, in embodiments where the single dose is fully mixed within the container, the single dose of the injectable medical formulation 100 is administered, by either directly injecting from the single container, or first withdrawing and then injecting from the single container 102. In other words, in embodiments having the formulation held within a vial, the medical personnel will withdraw and then inject. Alternatively, in embodiments wherein the formulation is already held within a syringe, the medical personnel will merely inject the formulation.

As is thus described, a selected formulation 100 is packed into the single container 102. In some embodiments, the single container 102 combines each of the medications into a pre-mixed dose. In other words, the medications are all added and mixed together such that only one withdraw or injection of the dose is needed for administration. Embodiments may include a single dose glass ampule, a single dose vial, or a single dose glass or synthetic syringe. Accordingly, in these embodiments, the medical personnel will make one withdraw for administering the formulation 100 to the patient or merely inject the formulation directly from the single container. Container 102 will be specifically selected based on a volume of the formulation. Those skilled in the art will appreciate that the volume may vary based on the predetermined amounts of each medication, however, most embodiments will have a volume of 3 mL or less, and even more embodiments will have a volume of 5 mL or less. Accordingly, container 102 will be selected to hold the appropriate volume.

Alternative embodiments include the single container 102 having separated chambers within the container. In such embodiments, one or more of the medications are separated via the separate chambers, yet still packaged together into the single container 102. Each chamber may hold a fixed amount of the associated medication, and therefore little or no measuring is required before administration. In other words, the medical personnel would obtain the single container 102, mix the medications held in the separate containers together (e.g., by removing or puncturing the sealing material or injecting the medications directly from the container), and then proceed with administration. In some embodiments, the opioids 112 may be mixed together in one chamber, while the local anesthetic 110 is held in a second chamber. Accordingly, the contents of only two chambers may be required to be mixed prior to administration. In other embodiments, the multi-chambered container may hold two or more medications separately, and is configured to combine the medications at the point of injection such that the medications are combined during administration, requiring no additional mixing step. And yet in additional embodiments, the multi-chambered container may include pre-measured amounts of opioids 112, while including an adjustable dosage of the local anesthetic 110, allowing for the medical personnel to adjust the amount of local anesthetic 110 as needed or desired at the time of injection.

The process of administering the formulation 100 is simplified from conventional processes as explained above. Specifically, the process of the invention enables execution of a treatment in minimal steps and allowing for little to no measuring or mixing of medications. Accordingly, the risk for human error is reduced, as well as the time necessary for the administration. Human error in this specialized practice can result in infection and or misdoing leading to severe morbidity and even mortality. In addition, this conventional process results in wasted medications, specifically wasted narcotics which are highly regulated due to the risk of diversion and misuse. The wasting process is a specialized, time-consuming process fraught with error and opportunity for diversion.

Those skilled in the art will appreciate that the specific formula for the injectable medical formulation 100 may vary and change as technology and research advance. Further, specific amounts of each medication are selected based on factors, such as dose (mg), baricity, site of injection (ie: lumbar spine), and patient position. Accordingly, not every formulation is the same, but rather may be created based on one or more factors and circumstances. Some contemplated embodiments are discussed below, including exemplary ranges for each medication. An artisan will select and combine the exact amounts based on the intended end purpose of the formulation.

In general, and as discussed above, each formulation will include the local anesthetic 110 in a hyperbaric or isobaric formulation. The local anesthetic 110 is selected from a group consisting of: 0.75% hyperbaric bupivacaine, 0.5% hyperbaric bupivacaine, 1.5% mepivacaine, 0.5% ropivacaine, 0.5% bupivacaine isobaric, 2% mepivacaine, and 0.5% hyperbaric ropivacaine. Those skilled in the art will appreciate that hyperbaric bupivacaine is the most common local anesthetic utilized for spinal anesthesia. However, other local anesthetic medications may be utilized and selected based on medical shortages, indication for shorter duration thus allowing for same-day discharge and earlier return of ambulation/bladder function, as well as for technology changes, or other reasons.

Medications added to the local anesthetic 110 are referred to as adjuvant medications. These can include the one or more opioids 112, the highly selective alpha-2 adrenergic receptor agonist 114, as well as one or more additional adjuvant medications 116. These medications are used to increase the quality and duration of the administered anesthetic and for providing pain management post medical procedure.

The one or more opioids 112 generally include at least one short acting opioid such as fentanyl at a concentration of 50 mcg/mL and one long-acting opioid such as morphine at a concentration of 0.5 mg/mL. An alternative short acting opioid is sufentanil and an alternative long-acting opioid is hydromorphone. Other alternative opioids or alternative concentrations may additionally or alternately be utilized as is known to those of skill in the art.

The one or more optional adjuvant medications 116 may be selected from a group consisting of: dexmedetomidine, clonidine, and epinephrine, combinations thereof, and the like. Clonidine and dexmedetomidine are examples of highly selective alpha-2 adrenergic receptor agonists which may be used according to the invention.

Tables A, B, and C below demonstrate proposed formulations having example ranges for each medication. Specifically, Table A demonstrates proposed formulations for use in cesarean sections. Table B demonstrates proposed formulations for use in spinal anesthesia after a failed labor epidural. Table C demonstrates proposed formulations for use in orthopedic surgery in adults for lower limb surgery where spinal anesthetic is appropriate and for general or for gynecologic surgeries where spinal anesthetic is deemed appropriate. Again, those skilled in the art will appreciate that selections of exact amounts of each medication are circumstantial based on factors such as medical procedure and patient physical data.

TABLE A
Injectable Medical Formulations for Cesarean Sections.

| | | | Adjuvant |
|---|---|---|---|
| | | | Medication(s) |
| | | | specifically |
| Local | Short Acting | Long Acting | prepared for |
| Anesthetic | Opioid | Opioid | neuraxial use. |
| 0.75% | Fentanyl | Morphine | Dexmedetomidine |
| hyperbaric | (50 mcg/mL) | (0.5 mg/mL) | (5-25 mcg) |
| bupivacaine | 5-50 mcg | 50-300 mcg | Clonidine |
| 10.5-12.75 mg | (0.1-1 mL) | (0.1-0.6 mL) | (5-25 mcg) |
| (1.4-1.7 mL) | Sufentanil | Hydromorphone | Epinephrine |
| | (5-20 mcg) | (25-100 mcg) | (5-250 mcg) |
| 0.5% | Fentanyl | Morphine | Dexmedetomidine |
| hyperbaric | (50 mcg/mL) | (0.5 mg/mL) | (5-25 mcg) |
| bupivacaine | 5-50 mcg | 50-300 mcg | Clonidine |
| 10.5-12.5 mg | (0.1-1 mL) | (0.1-0.6 mL) | (5-25 mcg) |
| (2.1-2.5 mL) | Sufentanil | Hydromorphone | Epinephrine |
| | (5-20 mcg) | (25-100 mcg) | (5-250 mcg) |

Table A demonstrates broad ranges of exemplary formulations used in cesarean sections. Specifically, a first formulation comprises 10.5-12.75 mg of 0.75% hyperbaric bupivacaine, 5-50 mcg of fentanyl at a concentration of 50 mcg/mL, and 50-300 mcg of morphine at a concentration of 0.5 mg/mL. An alternative formulation comprises 10.5-12.5 mg of 0.5% hyperbaric bupivacaine, 5-50 mcg of fentanyl at a concentration of 50 mcg/mL, and 50-300 mcg of morphine at a concentration of 0.5 mg/mL. To either formulation, one or more additional adjuvant medications may be added, including one or more of: dexmedetomidine in an amount of between approximately 5-25 mcg, clonidine in an amount of between approximately 5-25 mcg, and epinephrine in an amount of between approximately 5-250 mcg. As shown, sufentanil may be added as a replacement for fentanyl in an amount of between approximately 5-20 mcg, while hydromorphone may be added as a replacement for morphine in an amount of between approximately 25-100 mcg.

TABLE B
Injectable Medical Formulations for use in spinal
anesthesia after a failed labor epidural.

| | | | Adjuvant |
|---|---|---|---|
| | | | Medication(s) |
| | | | specifically |
| Local | Short Acting | Long Acting | prepared for |
| Anesthetic | Opioid | Opioid | neuraxial use. |
| 0.75% | Fentanyl | Morphine | Dexmedetomidine |
| hyperbaric | (50 mcg/mL) | (0.5 mg/mL) | (5-25 mcg) |
| bupivacaine | 10-20 mcg | 50-150 mcg | Clonidine |
| 7.5 mg | (0.2-0.4 mL | (0.1-0.3 mL) | (5-25 mcg) |
| (1 mL) | Sufentanil | Hydromorphone | Epinephrine |
| | (5-20 mcg) | (25-100 mcg) | (5-250 mcg) |
| 0.5% | Fentanyl | Morphine | Dexmedetomidine |
| hyperbaric | (50 mcg/mL) | (0.5 mg/mL) | (5-25 mcg) |
| bupivacaine | 10-20 mcg | 50-150 mcg | Clonidine |
| 7.5 mg | (0.2-0.4 mL | (0.1-0.3 mL) | (5-25 mcg) |
| (1.5 mL) | Sufentanil | Hydromorphone | Epinephrine |
| | (5-20 mcg) | (25-100 mcg) | (5-250 mcg) |

Table B demonstrates broad ranges of exemplary formulations used in spinal anesthesia after a failed labor epidural. Specifically, a first formulation comprises approximately 7.5 mg of 0.75% hyperbaric bupivacaine, approximately 10-20 mcg of fentanyl at a concentration of 50 mcg/mL, and approximately 50-150 mcg of morphine at a concentration of 0.5 mg/mL. An alternative formulation comprises approximately 7.5 mg of 0.5% hyperbaric bupivacaine, approximately 10-20 mcg of fentanyl at a concentration of 50 mcg/mL, and approximately 50-150 mcg of morphine at a concentration of 0.5 mg/mL. To either formulation, one or more additional adjuvant medications may be added, including one or more of: dexmedetomidine in an amount of between approximately 5-25 mcg, clonidine in an amount of between approximately 5-25 mcg, and epinephrine in an amount of between approximately 5-250 meg. As shown, sufentanil may be added as a replacement for fentanyl in an amount of between approximately 5-20 mcg, while hydromorphone may be added as a replacement for morphine in an amount of between approximately 25-100 mcg.

TABLE C
Injectable Medical Formulation for use in orthopedic surgery in adults for
lower limb surgery where spinal anesthetic is appropriate and/or for general
or gynecologic surgeries where spinal anesthetic is deemed appropriate.

| | | | Adjuvant |
|---|---|---|---|
| Local | Short Acting | Long Acting | Medication(s)specifically |
| Anesthetic | Opioid | Opioid | prepared for neuraxial use |
| 0.75% | Fentanyl (50 mcg/mL) | morphine (0.5 mg/mL) | Dexmedetomidine (5-25 mcg) |
| hyperbaric | 5-50 mcg (0.1-1 mL) | 50-300 mcg (0.1-0.6 mL) | Clonidine (5-25 mcg) |
| bupivacaine (2 mL) | Sufentanil (5-20 mcg) | Hydromorphone (25-100 mcg) | Epinephrine (5-250 mcg) |
| 0.5% | Fentanyl (50 mcg/mL) | morphine (0.5 mg/mL) | Dexmedetomidine (5-25 mcg) |
| hyperbaric | 5-50 mcg (0.1-1 mL) | 50-300 mcg (0.1-0.6 mL) | Clonidine (5-25 mcg) |
| bupivacaine (3 mL) | Sufentanil (5-20 mcg) | Hydromorphone (25-100 mcg) | Epinephrine (5-250 mcg) |
| Mepivacaine | Fentanyl (50 mcg/mL) | morphine (0.5 mg/mL) | Dexmedetomidine (5-25 mcg) |
| 1.5% (3-4 mL) | 5-50 mcg (0.1-1 mL) | 50-300 mcg (0.1-0.6 mL) | Clonidine (5-25 mcg) |
| | Sufentanil (5-20 mcg) | Hydromorphone (25-100 mcg) | Epinephrine (5-250 mcg) |
| Ropivacaine | Fentanyl (50 mcg/mL) | morphine (0.5 mg/mL) | Dexmedetomidine (5-25 mcg) |
| 0.5% (2.5-4 mL) | 5-50 mcg (0.1-1 mL) | 50-300 mcg (0.1-0.6 mL) | Clonidine (5-25 mcg) |
| | Sufentanil (5-20 mcg) | Hydromorphone (25-100 mcg) | Epinephrine (5-250 mcg) |
| 0.5% | Fentanyl (50 mcg/mL) | morphine (0.5 mg/mL) | Dexmedetomidine (5-25 mcg) |
| bupivacaine | 5-50 mcg (0.1-1 mL) | 50-300 mcg (0.1-0.6 mL) | Clonidine (5-25 mcg) |
| isobaric | Sufentanil (5-20 mcg) | Hydromorphone (25-100 mcg) | Epinephrine (5-250 mcg) |
| (2-3 mL) | | | |
| Mepivacaine | Fentanyl (50 mcg/mL) | morphine (0.5 mg/mL) | Dexmedetomidine (5-25 mcg) |
| 2% (2-3 mL) | 5-50 mcg (0.1-1 mL) | 50-300 mcg (0.1-0.6 mL) | Clonidine (5-25 mcg) |
| | Sufentanil (5-20 mcg) | Hydromorphone (25-100 mcg) | Epinephrine (5-250 mcg) |
| Hyperbaric | Fentanyl (50 mcg/mL) | morphine (0.5 mg/mL) | Dexmedetomidine (5-25 mcg) |
| Ropivacaine | 5-50 mcg (0.1-1 mL) | 50-300 mcg (0.1-0.6 mL) | Clonidine (5-25 mcg) |
| 0.5% | Sufentanil (5-20 mcg) | Hydromorphone (25-100 mcg) | Epinephrine (5-250 mcg) |
| (2.5-4 mL) | | | |

Table C demonstrates broad ranges of exemplary formulations used in orthopedic surgery in adults for lower limb surgery where spinal anesthetic is appropriate or for general or gynecologic surgeries where spinal anesthetic is deemed appropriate. Here, each formulation comprises approximately 5-50 mcg of fentanyl at a concentration of 50 mcg/mL and approximately 50-300 mcg of morphine at a concentration of 0.5 mg/mL, with each formulation having a different local anesthetic. Specifically, the first formulation includes approximately 2 mL of 0.75% hyperbaric bupivacaine, the second includes approximately 3 mL of 0.5% hyperbaric bupivacaine, a third includes approximately 3-4 mL of 1.5% mepivacaine, a fourth includes approximately 2.5-4 mL of 0.5% ropivacaine, a fifth includes approximately 2-3 mL of 0.5% bupivacaine isobaric, a sixth includes approximately 2-3 mL of 2% mepivacaine, and a seventh includes approximately 2.5-4 mL of 0.5% hyperbaric ropivacaine. As shown, sufentanil may be added as a replacement for fentanyl in an amount of between approximately 5-20 mcg, while hydromorphone may be added as a replacement for morphine in an amount of between approximately 25-100 mcg.

Each formulation may also include one or more additional adjuvant medications, such as: dexmedetomidine in an amount of between approximately 5-25 mcg, clonidine in an amount of between approximately 5-25 mcg, and epinephrine in an amount of between approximately 5-250 mcg.

Although the above examples demonstrate the use of the formulation 100 for limited procedures, those skilled in the art will appreciate that the formulation 100 may also be useful and applicable to any procedure in which spinal anesthesia is appropriate.

In Tables D, E, and F below, narrowed and preferred ranges for formulations for various medical procedures are shown.

TABLE D
Preferred Ranges for Injectable Medical
Formulations for Cesarean Sections.

| | Short Acting | Long Acting |
|---|---|---|
| | Opioid | Opioid |
| | Fentanyl | Morphine |
| Local Anesthetic | (50 mcg/mL) | (0.5 mg/mL) |
| 0.75% hyperbaric bupivacaine | 10-20 mcg | 50-150 mcg |
| 11.25-12 mg | | |
| 0.5% hyperbaric bupivacaine | 10-20 mcg | 50-150 mcg |
| 11.25-12.5 mg | | |

Table D demonstrates preferred ranges of exemplary formulations used in cesarean sections. Specifically, a first formulation comprises approximately 11.25-12 mg of 0.75% hyperbaric bupivacaine, approximately 10-20 mcg of fentanyl at a concentration of 50 mcg/mL, and approximately 50-100 mcg of morphine at a concentration of 0.5 mg/mL. An alternative formulation comprises approximately 11.25-12.5 mg of 0.5% hyperbaric bupivacaine, approximately 10-20 mcg of fentanyl at a concentration of 50 mcg/mL, and approximately 50-150 mcg of morphine at a concentration of 0.5 mg/mL.

TABLE E
Preferred Ranges for Injectable Medical Formulations for
use in spinal anesthesia after a failed labor epidural.

| | Short Acting | Long Acting |
|---|---|---|
| | Opioid | Opioid |
| | Fentanyl | Morphine |
| Local Anesthetic | (50 mcg/mL) | (0.5 mg/mL) |
| 0.75% hyperbaric bupivacaine | 10-20 mcg | 50-150 mcg |
| 7.5 mg | | |
| 0.5% hyperbaric bupivacaine | 10-20 mcg | 50-150 mcg |
| 7.5 mg | | |

Table E demonstrates preferred ranges of exemplary formulations used in spinal anesthesia after a failed labor epidural. Specifically, a first formulation comprises approximately 7.5 mg of 0.75% hyperbaric bupivacaine, approximately 10-20 mcg of fentanyl at a concentration of 50 mcg/mL, and approximately 50-150 mcg of morphine at a concentration of 0.5 mg/mL. An alternative formulation comprises approximately 7.5 mg of 0.5% hyperbaric bupivacaine, approximately 10-20 mcg of fentanyl at a concentration of 50 mcg/mL, and approximately 50-150 mcg of morphine at a concentration of 0.5 mg/mL.

TABLE F
Preferred ranges for Injectable Medical Formulations for use
in orthopedic surgery in adults for lower limb surgery where
spinal anesthetic is appropriate or for general or gynecologic
surgeries where spinal anesthetic is deemed appropriate.

| | Short Acting | Long Acting |
|---|---|---|
| | Opioid | Opioid |
| | Fentanyl | morphine |
| Local Anesthetic | (50 mcg/mL) | (0.5 mg/mL) |
| 0.75% hyperbaric bupivacaine | 10 mcg-20 mcg | 50 mcg-150 mcg |
| 15 mg | | |
| 0.5% hyperbaric bupivacaine | 10 mcg-20 mcg | 50 mcg-150 mcg |
| 15 mg | | |
| Mepivacaine 1.5% 45-60 mg | 10 mcg-20 mcg | 50 mcg-150 mcg |
| Ropivacaine 0.5% 12.5-20 mg | 10 mcg-20 mcg | 50 mcg-150 mcg |
| 0.5% bupivacaine isobaric | 10 mcg-20 mcg | 50 mcg-150 mcg |
| 10-15 mg | | |
| Mepivacaine 2% 40-60 mg | 10 mcg-20 mcg | 50 mcg-150 mcg |
| 0.5% Ropivacaine hyperbaric | 10 mcg-20 mcg | 50 mcg-150 mcg |
| 12.5-20 mg | | |

Table F demonstrates preferred ranges of exemplary formulations used in orthopedic surgery in adults for lower limb surgery where spinal anesthetic is appropriate or for general or gynecologic surgeries where spinal anesthetic is deemed appropriate. Each preferred formulation having approximately 10-20 mcg of fentanyl at a concentration of 50 mcg/mL, and approximately 50-150 mcg of morphine at a concentration of 0.5 mg/mL. A first formulation further comprises approximately 15 mg of 0.75% hyperbaric bupivacaine. A second formulation further comprises approximately 15 mg of 0.5% hyperbaric bupivacaine. A third formulation comprises approximately 45-60 mg of 1.5% mepivacaine. A fourth formulation comprises approximately 12.5-20 mg of 0.5% ropivacaine. A fifth formulation comprises approximately 10-15 mg of 0.5% bupivacaine isobaric. A sixth formulation comprises approximately 40-60 mg of 2% mepivacaine. A seventh formulation comprises approximately 12.5-20 mg of 0.5% ropivacaine hyperbaric.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A combination of a container and an injectable medical formulation for use in spinal anesthesia as part of an obstetric procedure, the combination comprising:
the injectable medical formulation including:
a pre-determined amount of a local anesthetic, the local anesthetic being a hyperbaric bupivacaine;
a pre-determined amount of a short-acting opioid, the short-acting opioid being one of fentanyl or sufentanil; and
a pre-determined amount of a long-acting opioid, the long-acting opioid being one of morphine or hydromorphone;
the container being a single apparatus in which the injectable medical formulation is packaged and stored as a single dose prior to the obstetric procedure;
wherein the injectable medical formulation is packaged and stored within the container prior to a window of imminency of the obstetric procedure.

2. The combination of claim 1, wherein the injectable medical formulation is packaged and stored within the container to create the single dose prior to knowledge of a specific obstetric procedure for which the single dose will be used.

3. The combination of claim 1, wherein the container consists of a vial in which the single dose is stored prior to the obstetric procedure, the single dose stored in such a way that requires one withdrawal from the vial for administration to a patient.

4. The combination of claim 1, wherein the container consists of a syringe in which the single dose is stored prior to the obstetric procedure, the single dose stored in such a way that requires one injection into a patient for administration of the single dose.

5. The combination of claim 1, wherein the container comprises a plurality of chambers, each chamber holding one or more parts of the single dose, the container configured to facilitate mixing of the one or more parts to create the single dose prior to administration to a patient but without any measuring of the one or more parts.

6. The combination of claim 1, wherein the injectable medical formulation further comprises:
the pre-determined amount of the local anesthetic and the local anesthetic are selected from a group consisting of:
the local anesthetic being 0.75% hyperbaric bupivacaine and the predetermined amount of the local anesthetic being an amount between approximately 10.5 mg and 12.0 mg; and
the local anesthetic being 0.5% hyperbaric bupivacaine and the pre-determined amount of the local anesthetic being an amount between approximately 10.5 mg and 12.5 mg;
the pre-determined amount of the short-acting opioid and the short-acting opioid are selected from a group consisting of:
the short-acting opioid being fentanyl at a concentration of 50 mcg/mL and the pre-determined amount of the short-acting opioid being an amount between approximately 5 mcg and 50 mcg; and
the short-acting opioid being sufentanil and the pre-determined amount of the short-acting opioid being an amount between approximately 5 mcg and 20 mcg;
the pre-determined amount of the long-acting opioid and the long-acting opioid are selected from a group consisting of:
the long-acting opioid being morphine at a concentration of 0.5 mg/mL and the pre-determined amount of the long-acting opioid being an amount between approximately 50 mcg and 300 mcg; and
the long-acting opioid being hydromorphone and the pre-determined amount of the long-acting opioid being an amount between approximately 25 mcg and 100 mcg;
wherein the obstetric procedure is a cesarian section.

7. The combination of claim 6, wherein the injectable medical formulation further comprises one or more adjuvant medications, the one or more adjuvant medications being added to the injectable medical formulation at pre-determined amounts, the one or more adjuvant medications in pre-determined amounts being selected from a group consisting of:
an amount between 5 mcg and 25 mcg of dexmedetomidine;
an amount between 5 mcg and 25 mcg of clonidine; and
an amount between 5 mcg and 250 mcg of epinephrine.

8. The combination of claim 1, wherein the injectable medical formulation further comprises:
the pre-determined amount of the local anesthetic and the local anesthetic are selected from a group consisting of:
the local anesthetic being 0.75% hyperbaric bupivacaine and the predetermined amount of the local anesthetic being an amount between approximately 11.25 mg and 12 mg; and
the local anesthetic being 0.5% hyperbaric bupivacaine and the pre-determined amount of the local anesthetic being an amount between approximately 11.25 mg and 12.5 mg;
the short-acting opioid is fentanyl at a concentration of 50 mcg/mL and the pre-determined amount of the short-acting opioid is an amount between approximately 10 mcg and 20 mcg; and
the long-acting opioid is morphine at a concentration of 0.5 mg/mL and the predetermined amount of the long-acting opioid is an amount between approximately 50 mcg and 150 mcg; and
wherein the obstetric procedure is a cesarian section.

9. The combination of claim 1, wherein the injectable medical formulation further comprises:
the pre-determined amount of the local anesthetic and the local anesthetic are selected from a group consisting of:
the local anesthetic being 0.75% hyperbaric bupivacaine and the predetermined amount of the local anesthetic being approximately 7.5 mg; and
the local anesthetic being 0.5% hyperbaric bupivacaine and the pre-determined amount of the local anesthetic being an approximately 7.5 mg;
the pre-determined amount of the short-acting opioid and the short-acting opioid are selected from a group consisting of:
the short-acting opioid being fentanyl at a concentration of 50 mcg/mL and the pre-determined amount of the short-acting opioid being an amount between approximately 10 mcg and 20 mcg; and
the short-acting opioid being sufentanil and the pre-determined amount of the short-acting opioid being an amount between approximately 5 mcg-20 mcg;
the pre-determined amount of the long-acting opioid and the long-acting opioid are selected from a group consisting of:
the long-acting opioid being morphine at a concentration of 0.5 mg/mL and the pre-determined amount of the long-acting opioid being an amount between approximately 50 mcg and 150 mcg; and
the long-acting opioid being hydromorphone and the pre-determined amount of the long-acting opioid being an amount between approximately 25 mcg and 100 mcg;
wherein the obstetric procedure is a procedure after a failed labor epidural.

10. The combination of claim 9, wherein the injectable medical formulation further comprises one or more adjuvant medications, the one or more adjuvant medications being added to the injectable medical formulation at pre-determined amounts, the one or more adjuvant medications in pre-determined amounts being selected from a group consisting of:
an amount between 5 mcg and 25 mcg of dexmedetomidine;
an amount between 5 mcg and 25 mcg of clonidine; and
an amount between 5 mcg and 250 mcg of epinephrine.

11. The combination of claim 1, wherein the injectable medical formulation further comprises:
the pre-determined amount of the local anesthetic and the local anesthetic are selected from a group consisting of:
the local anesthetic being 0.75% hyperbaric bupivacaine and the predetermined amount of the local anesthetic being approximately 7.5 mg; and
the local anesthetic being 0.5% hyperbaric bupivacaine and the pre-determined amount of the local anesthetic being approximately 7.5 mg;
the short-acting opioid is fentanyl at a concentration of 50 mcg/mL and the pre-determined amount of the short acting opioid is an amount between approximately 10 mcg and 20 mcg; and
the long-acting opioid is morphine at a concentration of 0.5 mg/mL and the predetermined amount of the long-acting opioid is an amount between approximately 50 mcg and 150 mcg;
wherein the obstetric procedure is a procedure after a failed labor epidural.

12. A combination of a container and an injectable medical formulation for use in spinal anesthesia as part of an orthopedic procedure, the combination comprising:
the injectable medical formulation including:
a pre-determined amount of a local anesthetic;
a pre-determined amount of a short-acting opioid, the short-acting opioid being one of fentanyl or sufentanil; and
a pre-determined amount of a long-acting opioid, the long-acting opioid being one of morphine or hydromorphone;
the container being a single apparatus in which the injectable medical formulation is packaged and stored as a single dose prior to the orthopedic procedure;
wherein the injectable medical formulation is packaged and stored within the container prior to a window of imminency of the orthopedic procedure.

13. The combination of claim 12, wherein the injectable medical formulation is packaged and stored within the container to create the single dose prior to knowledge of a specific orthopedic procedure for which the single dose will be used.

14. The combination of claim 12, wherein the container consists of a vial in which the single dose is stored prior to the orthopedic procedure, the single dose stored in such a way that requires one withdrawal from the vial for administration to a patient.

15. The combination of claim 12, wherein the container consists of a syringe in which the single dose is stored prior to the orthopedic procedure, the single dose stored in such a way that requires one injection into a patient for administration of the single dose.

16. The combination of claim 12, wherein the container comprises a plurality of chambers, each chamber holding one or more parts of the single dose, the container configured to facilitate mixing of the one or more parts to create the single dose prior to administration to a patient but without any measuring of the one or more parts.

17. The combination of claim 12, wherein the injectable medical formulation further comprises:
the pre-determined amount of the local anesthetic and the local anesthetic being selected from a group consisting of:
the local anesthetic being 0.75% hyperbaric bupivacaine and the pre-determined amount of the local anesthetic being approximately 2 mL;
the local anesthetic being 0.5% hyperbaric bupivacaine and the pre-determined amount of the local anesthetic being approximately 3 mL;
the local anesthetic being 1.5% mepivacaine and the pre-determined amount of the local anesthetic being an amount between approximately 3 mL and 4 mL;
the local anesthetic being 0.5% ropivacaine and the pre-determined amount of the local anesthetic being an amount between approximately 2.5 mL and 4 mL;
the local anesthetic being 0.5% bupivacaine isobaric and the pre-determined amount of the local anesthetic being an amount between approximately 2 mL and 3 mL;
the local anesthetic being 2% mepivacaine and the pre-determined amount of the local anesthetic being an amount between approximately 2 mL and 3 mL; and
the local anesthetic being 0.5% hyperbaric ropivacaine and the pre-determined amount of the local anesthetic being an amount between approximately 2.5 mL and 4 mL;
the pre-determined amount of the short-acting opioid and the short-acting opioid being selected from a group consisting of:
the short-acting opioid being fentanyl at a concentration of 50 mcg/mL and the pre-determined amount of the short-acting opioid being an amount between approximately 5 mcg and 50 mcg; and
the short-acting opioid being sufentanil and the pre-determined amount of the short-acting opioid being an amount between approximately 5 mcg and 20 mcg;
the pre-determined amount of the long-acting opioid and the long-acting opioid being selected from a group consisting of:
the long-acting opioid being morphine at a concentration of 0.5 mg/mL and the pre-determined amount of the long-acting opioid being an amount between approximately 50 mcg and 300 mcg; and
the long-acting opioid being hydromorphone and the pre-determined amount of the long-acting opioid being an amount between approximately 25 mcg and 100 mcg.

18. The combination of claim 17, wherein the injectable medical formulation further comprises one or more adjuvant medications, the one or more adjuvant medications being added to the injectable medical formulation at pre-determined amounts, the one or more adjuvant medications in pre-determined amounts being selected from a group consisting of:
an amount between 5 mcg and 25 mcg of dexmedetomidine;
an amount between 5 mcg and 25 mcg of clonidine; and
an amount between 5 mcg and 250 mcg of epinephrine.

19. The combination of claim 12, wherein the injectable medical formulation further comprises:
the pre-determined amount of the local anesthetic and the local anesthetic are selected from a group consisting of:
the local anesthetic being 0.75% hyperbaric bupivacaine and the predetermined amount of the local anesthetic being approximately 15 mg;
the local anesthetic being 0.5% hyperbaric bupivacaine and the pre-determined amount of the local anesthetic being approximately 15 mg;
the local anesthetic being 1.5% mepivacaine and the pre-determined amount of the local anesthetic being an amount between approximately 45 mg and 60 mg;
the local anesthetic being 0.5% ropivacaine and the pre-determined amount of the local anesthetic being an amount between approximately 12.5 mg and 20 mg;
the local anesthetic being 0.5% bupivacaine isobaric and the pre-determined amount of the local anesthetic being an amount between approximately 10 mg and 15 mg;
the local anesthetic being 2% mepivacaine and the pre-determined amount of the local anesthetic being an amount between approximately 40 mg and 60 mg; and
the local anesthetic being 0.5% hyperbaric ropivacaine and the pre-determined amount of the local anesthetic being an amount between 12.5 mg and 20 mg;
the short-acting opioid is fentanyl at a concentration of 50 mcg/mL and the pre-determined amount of the short-acting opioid is an amount between approximately 10 mcg and 20 mcg; and
the long-acting opioid is morphine at a concentration of 0.5 mg/mL and the predetermined amount of the long-acting opioid is an amount between approximately 50 mcg and 150.

20. A process of packaging an injectable medical formulation for use in spinal anesthesia, the process comprising:
providing:
(i) a pre-determined amount of a local anesthetic;
(ii) a pre-determined amount of a short-acting opioid, the short-acting opioid being one of fentanyl or sufentanil; and
(ii) a pre-determined amount of a long-acting opioid, the long-acting opioid being one of morphine and hydromorphone;
packaging the predetermined amount of the local anesthetic, the pre-determined amount of the short-acting opioid, and the pre-determined amount of the long-acting opioid into a container as a single dose;
sealing the pre-determined amount of the local anesthetic, the pre-determined amount of the short-acting opioid, and the pre-determined amount of the long-acting opioid into the container; and
configuring the container for transport to a medical treatment facility for a later administration of the single dose as part of a medical procedure, the medical procedure being one of: (i) an obstetric procedure; or (ii) an orthopedic procedure.

21. The process of claim 20 comprising:
executing the packaging and sealing of the pre-determined amount of the local anesthetic, the pre-determined amount of the short-acting opioid, and the pre-determined amount of the long-acting opioid into the container, and the configuring of the container for transport at a location separate from the medical facility and prior to knowledge of the medical procedure.

22. The process of claim 20 comprising:
selecting a vial to serve as the container;
configuring the vial to require withdrawal from the vial for administration to a patient.

23. The process of claim 20 comprising:
providing a syringe to serve as the container.

24. The process of claim 20 comprising:
including a plurality of chambers in the container;
holding one or more parts of the single dose in the each of the chambers such that an actuation of a syringe results in a mixing of the one or more parts to create the single dose prior to or during the administration to a patient without any measuring of the one or more parts.

25. The process of claim 20 comprising:
selecting hyperbaric bupivacaine to serve as the local anesthetic.
